(19) United States
(12) Patent Application Publication
(10) Pub. No.: US 2016/0060291 A1
(43) Pub. Date: Mar. 3, 2016

(54) PROCESS FOR RENATURATION OF POLYPEPTIDES

(71) Applicants: Archana Rajesh KRISHNAN, Maharashtra (IN); Sanjay Madhukar SONAR, Maharashtra (IN)

(72) Inventors: Archana Rajesh KRISHNAN, THANE (IN); Sanjay Madhukar SONAR, THANE (IN); Nikhil Sudhir GHADE, THANE (IN)

(73) Assignee: BioGenomics Limited, THANE WEST, MH (IN)

(21) Appl. No.: 14/388,892

(22) PCT Filed: Mar. 28, 2013
(86) PCT No.: PCT/IN2013/000204

§ 371 Date: Sep. 29, 2014

(30) Foreign Application Priority Data
Mar. 30, 2012 (IN) 1016/MUM/2012

(51) Int. Cl. C07K 1/113; C07K 14/535; C07K 14/62

(52) U.S. Cl. CPC C07K 1/1136; C07K 14/62; C07K 14/535; C07K 2319/21

(57) ABSTRACT

The invention relates to method of refolding of proteins from a solution containing the protein in predominantly misfolded, aggregated form. The method involves denaturation and reduction of the protein of interest. The denatured and reduced preparation is subjected to removal of reducing agent in denaturing condition and at low pH to prevent the misfolding of the protein. The protein preparation is subjected to refolding followed by removal of the refolding buffer components.

### CROSS REFERENCE TO RELATED APPLICATION

This application claims priority to the pending PCT application PCT/IN2013/000204, filed on 28 Mar. 2013. The pending PCT application PCT/IN2013/000204 is hereby incorporated by reference in its entireties for all of its teachings.

### FIELD OF THE INVENTION

The present invention relates to the field of protein Biochemistry. More specifically the invention relates to the field of refolding of proteins from the preparation containing proteins in the predominantly misfolded, aggregated form. A novel method is provided for refolding of protein where misfolded, aggregated proteins are unfolded by following specific sequence of steps and then refolded to achieve higher yield for properly refolded biologically active form of protein by preventing formation of misfolded and aggregated form of protein. The process further involves treatment of refolded preparation for removal of refolding components.

### BACKGROUND OF THE INVENTION

Recombinant proteins are used as modern therapeutic agents. Such proteins include Human Insulin, Human growth hormone, Human granulocyte colony stimulating factor, etc. Said proteins are used for various therapeutic applications like controlling blood glucose level, treatment of abnormal height, and increase in the granulocyte population respectively. Production of recombinant proteins is carried out using Recombinant DNA (rDNA) technology. The process involves cloning of the gene of interest into a suitable vector, Introduction of said recombinant vector into a host cell, promoting production of recombinant protein in the host cell followed by isolation and purification of the protein of interest from the host cell. Host cell used for the production can be a Eukaryotic host cell like Chinese hamster ovary (CHO) cells or Prokaryotic host cell like Escherichia coli cells.

Protein expressed using eukaryotic cells are expressed in their biologically active form because of the cellular machinery available in the host cells. But the relatively low expression levels combined with high cost of culture media and expensive quality control programs makes it generally more expensive to produce recombinant proteins in animal cells.

Protein expression in E. coli is usually faster than in Eukaryotic cells. This is due to E. coli fast growth rate, good protein production rate and undemanding growth conditions. The cost of production of proteins in E. Coli is low when compared to the Mammalian system.

The E. coli system provides a high expression levels and allows use of high density expression techniques, using this system gram amount of the desired proteins can be obtained per liter of the fermentation broth, however, there is a problem associated with over expression of foreign proteins in E. coli. Often these desired polypeptides synthesized in prokaryotic host cells are not in the correctly folded, native forms. Instead of the biologically active product, aggregated inclusion bodies are formed. These inclusion bodies are mostly biologically inactive and are insoluble in non-denaturing buffer system.

Inclusion bodies are treated to achieve the protein in a biologically active form. The treatment involves solubilizing the inclusion bodies in denaturing conditions followed by refolding of the proteins. Various factors affect the process of protein refolding like concentration at which refolding of a particular protein is carried out, composition and pH of the refolding buffer, temperature, time and ionic strength, and concentration of denaturing agent and reducing agent. While some proteins having less complexity in the design, refold efficiently in simple buffer solutions, other may need sophisticated process design and cost intensive additives, additional steps to achieve successful refolding of the protein at desired efficiency levels (Lange, C. and Rudolph, R. 2008. Production of Recombinant Proteins by In Vitro Folding. Protein Science Encyclopedia. 1245-1280).

Efficiency of the refolding process is very important factor which determines the overall yield of the protein purified in the biologically active form. During the refolding process, the unfolded molecules forms the native and folded active form of the target protein, however, the reaction is in competition with ability of the unfolded form to form multimers and aggregates. The unfolded molecule to be refolded can form improper intramolecular interaction or intermolecular interaction. This can reduce the percentage of protein which is properly folded (yield of refolding). The efficiency of the overall protein purification process is directly dependent on the amount of protein present in the desirable folded form. Other parameters affecting the refolding efficiency are presence of denaturing and reducing agents in the reaction buffer.

Various processes are described in the art for carrying out industrial scale refolding of the protein. The commonly followed methods involves the solubilization, denaturation and unfolding of the protein molecule present in inclusion bodies. This is most commonly done by utilization of strong chaotropic agents for denaturing of the protein. If the protein to be refolded consists of disulfide bonds then breaking of such disulfide bonds would require addition of reducing agent. The denatured and reduced protein is subjected to refolding by reducing the concentration of denaturant leading the unfolded molecule to refold. This method provides very low yield as the presence of reducing agent in the protein preparation interferes with the formation of proper disulfide bond. This provides very low yield for the refolding of the protein. Alternative to this method is the removal of the reducing agent prior to subjecting the reduced protein preparation to refolding. This method does not prevents formation of unrequired disulfide bonds. The overall yield achieved for the refolding is very less due to loss of protein due to aggregation and misfolding.

Therefore, it is clear that a need continues to exist for providing a method for refolding of the proteins which can provide better yield for refolding by preventing formation of misfolded and aggregated proteins during the refolding step. The process should be scalable and hence can be useful for industrial level refolding of proteins.

### SUMMARY OF THE INVENTION

It has been found, after a series of experiments, that the removal of reducing agent from the reduced and denatured protein preparation if carried out at low pH and in denaturing conditions, prevents the disulfide bond formation. When such protein preparation which is devoid of any reducing agent is subjected to refolding step by dilution in refolding buffer, the refolding yield achieved is more as compared to the yield achieved using conventional methods.

The invention provides a method for refolding of the protein from the solution of predominantly unfolded, misfolded form of the protein to obtain the protein solution containing protein in significantly folded form. The specific sequence of the steps and the specific conditions maintained during those steps provide better yield for refolding of the protein.

Accordingly, in one embodiment of this invention the suspension containing the protein in predominantly unfolded, misfolded form is denatured, affinity purified and then subjected to reduction. The denatured, affinity purified and reduced sample is then subjected to low pH and the removal of reducing agent is carried out at low pH and in denaturing condition. This is followed by refolding of the sample by subjecting it to dilution in refolding buffer at appropriate concentration to allow optimum refolding of protein. The refolded protein preparation is then treated to remove the refolding agent and denaturing agent with appropriate buffer at appropriate conditions.

In another embodiment the preparation containing the protein in predominantly misfolded and unfolded form is denatured, reduced and then subjected to low pH. The removal of the reducing agent is carried out at low pH and the suspension is subjected to refolding at appropriate concentration of refolding buffer sufficient to achieve optimum refolding of the protein of interest. The refolded protein preparation is then treated to remove the refolding agent and denaturing agent.

Hence, a significant feature of the method used in the present invention is that it makes use of step which separates the reducing agent from the protein preparation without allowing disulphide bond formation in the protein molecule. This is achieved by carrying out removal of the reducing agent at low pH which prevents disulfide bond formation thereby preventing aggregation and concomitant misfolding of proteins. The removal of reducing agent is carried out in the presence of chaotropic agent so as to maintain the protein in solubilized state as well as in the denatured state. The said protein preparation when subjected to refolding provides better yield even at low concentration of redox shufflers.

### BRIEF DESCRIPTION OF DRAWINGS

FIG. 1: Process flow chart for refolding of protein.

FIG. 2: RP-HPLC Analysis of Denatured, Reduced and exchanged fusion protein linked Human proinsulin preparation. 150 μg of fully reduced and denatured fusion protein linked proinsulin was injected on C18 column. Fully reduced and denatured fusion protein linked human proinsulin (dialyzed against 8 M urea, pH 3.0) shows the formation of a single sharp peak at 32.753 min. The peaks marked 1, 2 and 3 have no absorbance in the ultraviolet region.

FIG. 3: RP-HPLC monitoring of folding of fusion protein linked Human Proinsulin at various time intervals. RP-HPLC analysis of refolding reaction at 0^{th} hour. 150 μg of fully reduced fusion protein linked human proinsulin was injected on a C18 column. The fully reduced fusion protein linked human proinsulin was subjected to refolding as per the operational model and the reaction was analyzed at the 0^{th} hour. The chromatogram shows the formation of an intermediate peak at 27.805 min. The peak marked 1, 2, 3, 4 and 5 have no absorbance in ultra violet region.

FIG. 4: RP-HPLC Analysis for calculation of refolding percentage achieved for refolding reaction carried out using varied concentration of redox constituents (refolding agents).

FIG. 5: SDS PAGE analysis of fusion protein linked Human proinsulin refolding carried out using various concentration of cysteine/cysteine RP-HPLC analysis of samples generated at different times for fusion protein linked human proinsulin folding shows the formation of peak labelled R corresponding to fully reduced and denatured fusion protein linked human proinsulin dialized against 8 M urea, pH 3.0 and observed at 32.7 min. An intermediate peak (I) is observed at 27.8 min for the aliquot taken at 0 hrs. The completely folded native form (N) of the molecule is observed at 25.3 min for the aliquot taken at 0.5 hrs.

FIG. 6: Silver stained SDS PAGE analysis of Fusion protein linked Human proinsulin refolding in the presence of varied concentrations of urea.

FIG. 7: RP-UFLC Analysis of refolding reaction of fusion protein linked Human proinsulin preparation carried out using refolding buffer consisting of 0.5 M-2.5M Urea

FIG. 8: RP-HPLC analysis of denatured, reduced and exchanged Fusion protein rhG-CSF.150 μg of Fully reduced and denatured fusion protein linked hG-CSF was injected on a C4 column. The reduced and dentured fusion protein linked hG-CSF (dialized against 8 M Urea, pH 3.0) shows the formation of a single sharp peak at 48.3 min.

FIG. 9: RP-HPLC monitoring of Fusion Protein linked hG-CSF monitored at different times. 150 μg of fully reduced and denatured Fusion protein linked hG-CSF was injected on a C4 column. The fully reduced and denatured Fusion protein linked hG-CSF (dialyzed against 8 M Urea, pH 3.0) shows the formation of a single sharp peak at 48.3 min.

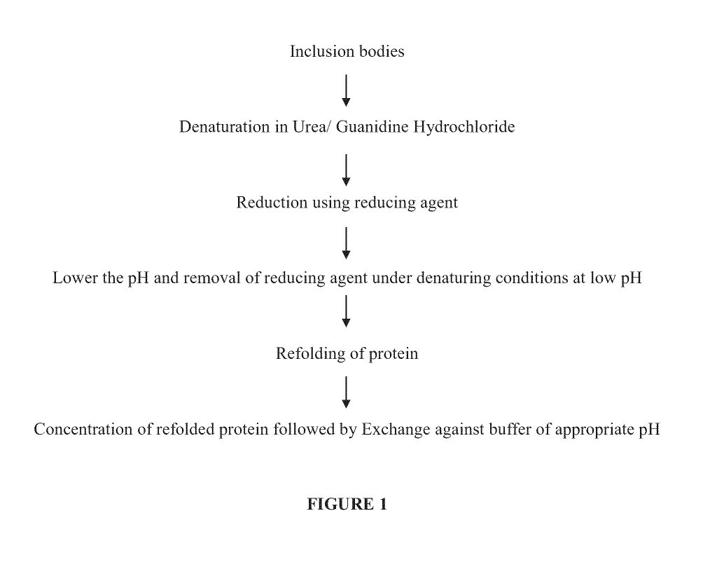

### DETAILED DESCRIPTION OF INVENTION

Accordingly the subject invention provides a method for obtaining a preparation of protein where substantial part of the protein is in a refolded form from a preparation comprising a protein in a predominantly aggregated, misfolded or unfolded form, a preparation of the protein where significant part of the protein in the preparation is refolded.

The subject invention also provides a method for refolding of proteins which provides high yield of biologically active refolded protein by preventing formation of misfolded and aggregated proteins during refolding step.

The subject invention also provides a method for refolding of proteins which provides higher yields for refolding of proteins and the said method incorporates the unit processes which are scalable to manufacturing level.

As is instrumented in the examples, the various embodiments of the invention shows that removal of the reducing agent from the protein preparation at denaturing conditions and at low pH without allowing disulfide bond formation, prevents formation of intermediate forms and subjecting this preparation to refolding provides better yields as compared to known methods. The sequence of steps used prior to refolding is responsible for providing optimum refolding efficiency.

“Denatured form”, in the meaning of the present invention, designates the biologically inactive, unfolded form of the expressed protein of interest, as obtained as an intermediate product during the production of proteins using recombinant DNA technology, e.g. as obtained after dissolving the inclusion bodies in denaturing agent.

“Denaturing agent/Chaotropic agent” in the meaning of the present invention, designates any chemical substance or combination thereof which can alter the secondary and tertiary structure of the protein. Commonly known denaturing agents include Urea, Guanidine hydrochloride, etc.

“Intermediate forms” or “intermediates” in the meaning of the present invention, designates all the forms that the protein passes through or achieves or can achieve between its denatured form and its reconstituted (refolded) native and biologically active state. The intermediates, which are biologically inactive or have a lower biological activity than the native protein, may form aggregates.

A “protein” in the meaning of the present invention is any protein, protein fragment or peptide that requires refolding upon recombinant expression in order to obtain such protein in its biologically active form.

“Monomeric form”, in the meaning of the present invention, designates the forms that the protein achieves after treatment with chaotropic agent followed by reducing agent and is a form in which the protein is substantially devoid of any disulfide bonds.

“Exchange” in the meaning of present invention is a process of treatment of a protein containing preparation with another buffer. The exchange can be carried out using techniques like ultrafiltration, dialysis, precipitation, etc.

“Refolded form”, in the meaning of the present invention, designated the forms that the protein achieves after formation of the proper disulfide bonds thereby achieving proper three dimensional conformation and the form of the protein which allows the protein to perform its biological function or show its biological activity.

“Refolding Agent”, in the meaning of the present invention is any chemical/substance or combination of chemicals/substances which can carry out disulfide bond formation in the protein thereby restoring the biologically active conformation of the protein.

“Reducing agent” in the meaning of the present invention is any substance/chemical or combination of chemical/substances that tends to bring about reduction by being oxidized and losing electrons thereby causing breakage of disulfide bonds in the protein.

“Refolding preparation” in the meaning of the present invention is any preparation comprising the mixture of protein to be refolded and refolding buffer (containing refolding agent). The preparation may consist of some additional components like host cell impurities, related proteins, peptides, insoluble impurities, etc.

The protein expressed in the inclusion bodies is in the aggregated form. The said protein need to be converted to its biologically active form. The aggregated form of the protein can be obtained by expression of the protein in Escherichia coli. Any strain of E. coli may be used to produce protein useful for refolding using the method of the invention, so long as that strain is capable of expression of heterologous proteins, one preferred strain is E. coli strain BL21. Other strains of E. coli which can be used for production of inclusion bodies include are Origami, GI724 or BL21DE3, etc. The protein expressed in the inclusion bodies of these strains can be used to produce protein useful for refolding using the method of the invention.

The method of the present invention can be used for refolding of protein expressed in the bacteria. The proteins can be expressed in bacteria by using various processes known to the person skilled in the art. The protein to be produced may be a modified protein having difference of one or more amino acids than the native protein. For Example the protein can be Insulin Lispro having modification at B28^{th} and B29^{th} position of Human Insulin. The protein may contain some additional amino acids or may contain some deletions in the amino acids. The protein may also contain N-Terminal or/and C-Terminal tags of amino acid sequence useful for affinity purification of the protein. The preferred affinity tag is any tag which allows affinity purification under denaturing condition. One such tag is a 6× Histidine tag which allows the purification of the molecule using Immobilized Metal-Ion Affinity Chromatography (IMAC) under denaturing condition. The 6× histidine tag allows the separation of the protein of interest from other proteins under denaturing conditions. The separation of protein of interest from other proteins can be carried out in early stages thereby preventing the presence of other proteins during refolding step. The absence of other proteins during refolding step allows achievement of higher refolding efficiency. Other tags which can be used for affinity purification include GST tag, MBT tag, CBP tag, etc. In cases where affinity tag used is a tag like GST which does not allow separation of protein linked to the tag in denatured condition, affinity purification will be carried out after refolding step.

The modified or unmodified nucleotide sequence which encodes protein to be expressed may be inserted into a plasmid suitable for transformation and expression of said heterologous proteins in bacteria. Any bacterial expression plasmid may be used which is capable of directing the expression of a heterologous protein in the bacteria chosen. Some bacterial plasmids which can be used for expression of the protein include PET28a, PET38b, Plex vector series, PUC vectors, etc. Acceptable species of bacteria include E. coli. Suitable expression plasmids for this species are well known to the person skilled in the art. For production of protein in bacteria, a suitable vector having suitable restriction sites can be used for cloning the gene of interest. The bacterial expression plasmid may be transformed into a competent bacterial cell. The methods for introducing the plasmid into a bacterial cell more specifically E. coli are well known to the person skilled in the art some are Heat shock method, electroporation, Transformation, etc. Successful transformants are selected by selection strategy depending upon the type of vector used for cloning the gene of interest and the characteristics of the host cells. The general procedure involves growing the cells on the medium containing an appropriate drug when drug resistance is used as the selective pressure, or for growth on medium which is deficient in an appropriate nutrient when auxotrophy is used as the selective pressure. Expression of the heterologous protein may be optimized using known methods. The protein thus obtained will be present in insoluble, retractile inclusion bodies which can be isolated by disruption and centrifugation of the cells. The method of isolation and purification of the inclusion bodies from host cells are well known to the person skilled in the art.

The inclusion bodies thus obtained may be solubilized using a denaturing concentration of chaotropic agent such as Guanidine Hydrochloride or Urea. The concentration of Urea can be 4-8 M and Guanidine Hydrochloride will be 4-6 M. After solubilization using a chaotropic agent, a reducing agent such as B-mercaptoethanol, Cysteine, reduced glutathione, Dithiothreitol (DTT) or any other thiol group containing molecule is added with the denaturant. The solution can be incubated for appropriate time and at appropriate temperature to allow reduction of the protein to occur. This should ensure that the monomeric form of the protein is achieved. The optimum concentration of reducing agent required for carrying out full reduction of the protein can be determined as shown in example no 2. The form of incubated protein can be monitored using techniques known in the art like SDS PAGE, HPSEC (HPLC size exclusion chromatography), etc.

Prior to refolding or after refolding, the solubilized protein of interest may be further purified using known chromatographic methods such as Affinity chromatography, size exclusion chromatography or reverse phase high performance liquid chromatography, etc. The chromatography step is included in the process with an intention to purify the protein of interest from the mixture of proteins. The chromatography step prior to refolding can be desirable in some cases to achieve the better yield for refolding of the protein of interest. The chromatography step can be done after the denaturation step. The chromatography step can be done in a denaturing or non denaturing environment which will prevent the improper refolding of the protein of interest. The addition of reducing agent to the protein preparation can be done after the chromatography step. The denatured and reduced preparation of the protein is subjected to low pH prior to start of the removal of reducing agent. The pH may be lowered in the range of 1.5-3.5 using HCL. The low pH ensures that no disulfide bond formation is resulted during the process of removal of reducing agent. The incorporation of a diafiltration step against low pH denaturing buffer, without adjusting the pH of denatured and reduced protein solution to 1.5-3.5 would promote the formation of non-native disulphide bonds during pH transition, and thus the complete reduced and denatured state of the molecule cannot be maintained during and after the step of removal of reducing agent. This state of the molecule when subjected to renaturation would result into lower refolding efficiencies.

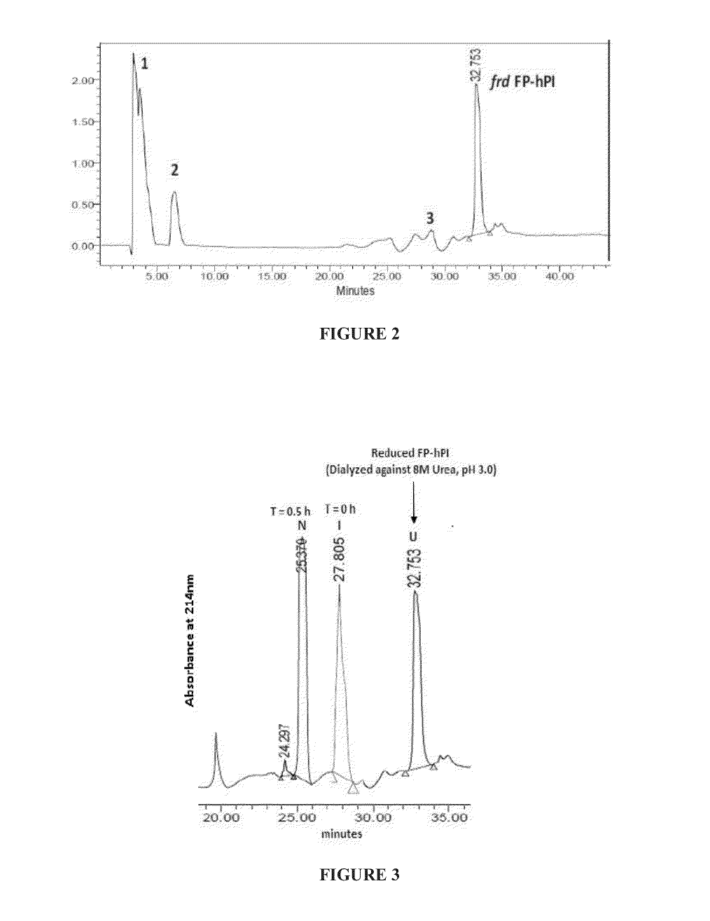

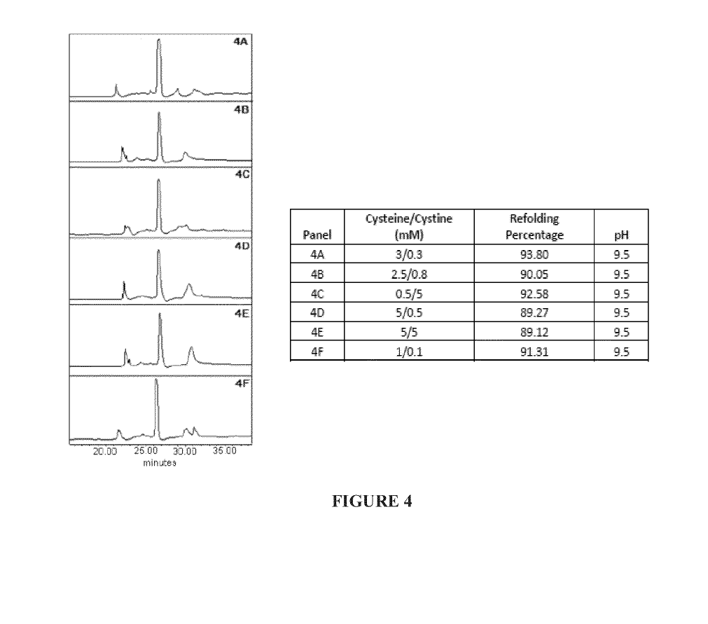

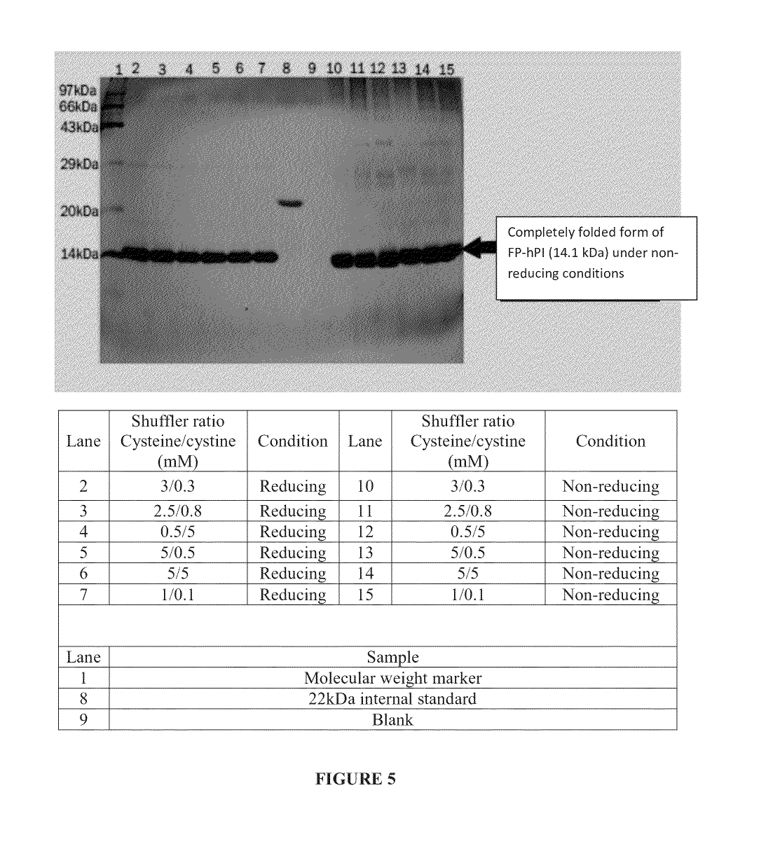

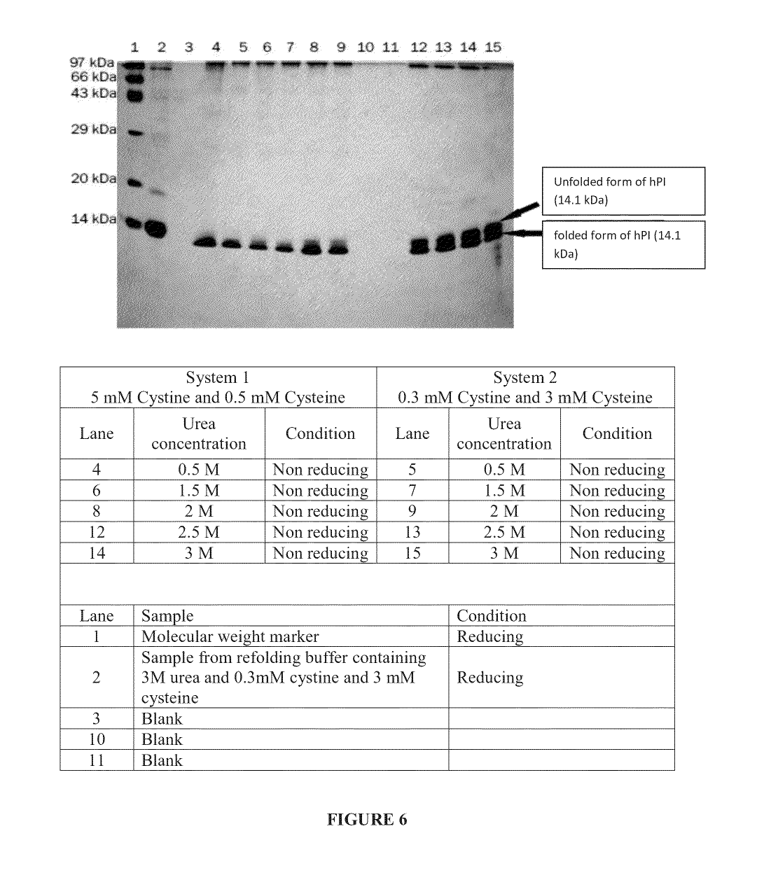

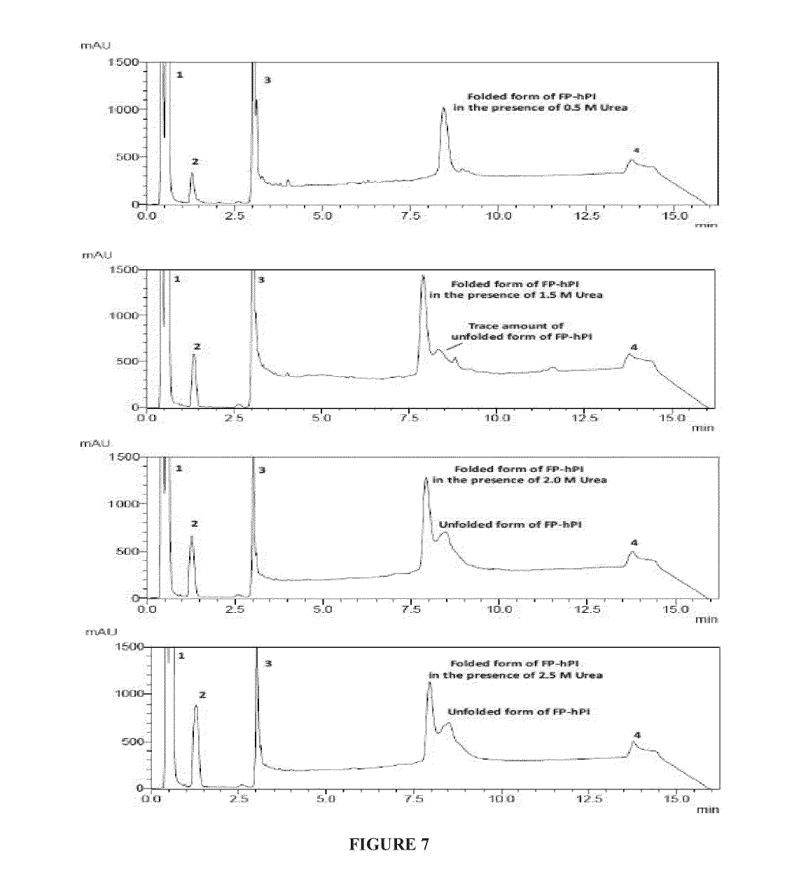

This is followed by the removal of the reducing agent under denaturing conditions and at low pH. The removal of the reducing agent can be carried out using dialysis, Ultra filtration, Size exclusion chromatography (SEC) against low pH denaturing buffer devoid of reducing agent. The buffer used for exchange shall be devoid of any reducing agent and shall ideally include same concentration of denaturing agent as present in the protein solution to be refolded. This will ensure that the denatured protein preparation does not form any intermediate form and retains its denatured form. The uniform state of the protein molecules can be confirmed by subjecting the denatured, reduced and exchanged protein solution for RP-HPLC analysis (FIG. 2). The preparation obtained after removal of reducing agent may then be subjected to refolding by diluting in appropriate volume of refolding buffer containing refolding agent to achieve dilution in the range 0.05 mg/ml-1.0 mg/ml for the protein to be refolded. The refolding agent can be cysteine/cystine, Reduced and oxidized glutathione, cysteamine/cystamine, DTT/GSSG and DTE/GSSG. For example, refolding buffer includes the following ingredients: Buffering composition for achieving pH of 8.5-11.0, varied concentration of refolding agents, Non denaturing concentration of chaotropic agent (0.1-3 M of Urea, 0.1-1.5 M Guanidine Hydrochloride). For Example, the refolding buffer is consists of 100 mM Tris-HCl (pH 9.5), 1 mM EDTA, 3 mM of cysteine, 0.3 mM of cystine and 1.5 M Urea. The impact of different redox constituents on the refolding yield of the protein can be determined as shown in FIGS. 4, 5 and example no 4. The refolding reaction can be monitored using various techniques known to the person skilled in the art. Some techniques include RP-HPLC, SDS-PAGE and Circular Dichroism. The optimum concentration of denaturing agent required in the refolding buffer for achieving optimum refolding efficiency for the protein can be determined as shown in Example 5 and FIGS. 6 and 7. Characteristic of the peak generated on the chromatogram for the desired protein of interest after the RP-HPLC analysis of the refolded sample can be analyzed to determine the optimum concentration of the urea required in the refolding buffer. This may optionally require (In case of molecules like insulin) subjecting the refolded protein to enzymatic digestion (Trypsin, CPB) and then carrying out RP-HPLC analysis to determine the pattern of the peptides generated after refolding of the protein carried out using various concentration of urea.

The refolded protein preparation can be treated to remove the refolding agents and denaturing agent/chaotropic agent. The removal of the denaturing agent and refolding agent can be preferably carried out using Ultrafiltration. Other techniques which can be used to remove these agents are known to the person skilled in the art, some are Size exclusion chromatography, dialysis, precipitation, etc. The removal is carried by exchange of the protein preparation against buffer of appropriate pH which is devoid of the refolding agent and the denaturing agent. The buffer may be of acidic pH or of Basic pH. For removal of the chaotropic agent and the refolding agent from the refolded preparation of protein containing free SH groups the buffer should be preferably of basic pH. For removal of the denaturing agent and the reducing agent from the preparation of the protein devoid of free SH group the buffer should be preferably of acidic pH. The basic buffer can be Tris pH 8.5-10.5 most preferably of pH 8.5 and acidic buffer can be Glycine pH 2-4 most preferably of pH 3. The basic buffer can be also selected from the group of Tris (hydroxylmethyl) aminomethane/Hydrochloric acid (pH 7.0-9.00), Sodium tetraborate/Hydrochloric acid (pH 8.1-9.2), Glycine/Sodium hydroxide (8.6-10.6), Sodium carbonate/Sodium hydrogen carbonate (pH 9.2 -10.8), Sodium tetraborate/Sodium hydroxide (pH 9.3-10.7), Sodium bicarbonate/Sodium hydroxide (pH 9.60-11.0), etc. The acidic buffer can also be selected from the group of Hydrochloric acid/Potassium chloride (pH 1.0-2.2), Glycine/Hydrochloric acid (pH 2.2-3.6), Potassium hydrogen phthalate/Hydrochloric acid (pH 2.2-4.0), Citric acid/Sodium citrate (pH 3.0-6.2), Sodium acetate/Acetic acid (pH 3.7-5.6). The refolded protein preparation can be concentrated to the range of 0.5 mg/ml-3 mg/ml prior to exchange against the buffer for removal of refolding agent. This will reduce the volume of buffer required for exchange of refolded protein preparation. The volume of the refolded protein preparation can be reduced by ultrafiltration.

The concentrated and desalted protein preparation can be further subjected to various purification steps depending upon the nature of the protein and the desired specification. The purification steps may include one or more chromatographic steps. The chromatography step may include affinity chromatography, size exclusion chromatography, reverse phase chromatography, etc or any combination thereof. The purification steps may also include an enzymatic cleavage step required for the removal of the affinity tag or the unrequired portion of the protein. For example the Polyhistidine tag linked to the protein sequence can be removed by using enzymes like Trypsin or CPB or enterokinase if the sites susceptible to these enzymes are included in the protein sequence or the protein construct. In production of proteins like Human Insulin, Insulin LysPro, Insulin Glargine, Insulin Aspart removal of the C chain peptide might be necessary. This can be achieved using enzymatic cleavage using enzymes known to the person skilled in the art. The treatment of the protein preparation subjected to such enzymatic treatment may involve use of one or more chromatographic steps to separate the protein of interest from the enzymatic digestion products.

The protein may undergo processes like lyophilization to achieve a lyophilized form of the protein. The various features and aspects of the present invention are illustrated with the help of following non-limiting examples.

### EXAMPLES

### Example 1

### Cloning and Expression of Recombinant Insulin

The cDNA of Human Proinsulin (hPI) linked to a sequence coding for affinity tag (6× histidine) was cloned in pBR 322 derived expression vector and the said vector was introduced into strain BL21 (DE3) of Escherichia coli. The fusion protein linked Human proinsulin was expressed in the inclusion bodies of the E. coli using conventional methods. The cell pellet was collected by centrifugation at 4° C. and washed with buffer containing 20 mM Tris (pH 8.0), 0.5% Triton X-100 and 2M Urea and the inclusion bodies were collected by centrifugation. The pellets and supernatants obtained during Inclusion Bodies isolation process were analyzed using SDS PAGE.

### Example 2

### Pretreatment of the Purified Fusion Protein Linked hPI

10 gm of isolated inclusion bodies were dissolved in 1 liter buffer containing 8.0 M Urea, 20 mM Tris (pH 8.5). The sample was then centrifuged at 8,000 rpm for 20 min at 4° C. The supernatant was collected and subjected to Immobilized Metal Ion Affinity Chromatography (IMAC) at room temperature. XK-16 column packed with chelating Sepharose fast flow media was connected to an AKTA Explorer 100 (Amersham Biosciences) chromatographic system was used for chromatography purification. The packed column was cleaned to remove impurities with 3 bed volumes of 1 M NaOH and by passing 4 bed volumes of purified water. The column was charged with 0.1 M NiSO₄ and was equilibrated with buffer-A consisting of 50 mM Tris (pH 8.5), 8.0 M urea and 500 mM NaCl. The sample was loaded on the column and the loosely bound proteins were removed by passing 3-4 bed volumes of Buffer A. The Non specific proteins were removed by passing 20 mM Tris (pH 8.5), 8 M Urea, 500 mM NaCl and 35 mM Imidazole. Fusion protein was eluted is the presence of 150 mM of Imidazole and 500 mM Imidazole.

Fully reduced and denatured fusion protein preparation was prepared by adding the 6 gm of purified fraction from IMAC to a 1.5 liter of buffer containing 100 mM Tris-HCL (pH 8.5), 1 mM ethylene diamine tetra acetic acid (EDTA) and 100 mM Dithiothretol (DTT). The sample was incubated at 37° C. for three hours. The optimum concentration of DTT for denaturing protein preparation was evaluated using varied amounts of DTT for carrying out reduction of denatured proinsulin and by analyzing the reduced samples on non-reducing conditions of silver stained SDS PAGE. The concentration of DTT used for carrying out reduction of the protein was 10-100 mM. The presence of dimeric and multimeric forms of fusion protein linked Human Proinsulin were identified based on their mobility differences. The multimeric form of the molecule is present in the form of bands at the top of SDS PAGE, around the 97 kDa marker. A high percentage of multimeric and dimeric form of the molecule was observed when 5, 10 and 15 mM DTT was used. A gradual decrease was observed in multimeric and dimeric forms with increase in the amount of reducing agent. Complete reduction of the molecule was observed in the presence of 100 mM DTT.

20 mM Glycine was added to the reduced and denatured protein preparation and the pH was adjusted to 3.0 using HCL.

The low pH preparation was subjected to ultrafiltration using a membrane having a 5 kDa Molecular weight cutoff (MWCO) against a 10 liter solution containing 8.0 M urea, 20 mM Glycine (pH 3.0) to remove the reducing agent. The uniform state of molecule was confirmed by subjecting the denatured, reduced and exchanged sample to RP-HPLC analysis. The reduced and exchanged preparation (against 8 M urea, pH 3.0) shows the formation of a single sharp peak at 32.753 min. The peaks marked 1, 2 and 3 have no absorbance in the ultraviolet region (FIG. 2).

### Example 3

### Folding of Treated Fusion Protein Linked Human Proinsulin

The low pH protein preparation which was treated to remove reducing agent was subjected to refolding in a 20 liter refolding buffer consisting of 100 mM Tris-HCL (pH 9.5), 1 mM EDTA, 3 mM of Cysteine and 0.3 mM of Cystine and 1.5 M Urea. The refolding was performed at protein concentration of 0.3 mg/ml. The preparation was incubated at 4° C. for 0.5 h. For monitoring of refolding reaction, Aliquots from the refolding reaction of fusion protein linked recombinant Human Proinsulin (150 μg) were quenched by adding Trifluoroacetic acid to make pH lower than 3.0 and were injected onto a C18 reverse phase column. The solvents used for chromatography were Solvent A: 0.1% TFA; solvent B: 0.1% Trifluoroacetic acid and 99.9% Acetonitrile. The column was initially equilibrated with 100% A at 1 ml/min. The separation was performed by a linear gradient from 20% B to 100% B within 40 minutes on HPLC system equipped with a photodiode array. Protein peaks were detected at 214 nm (FIG. 3). The pH of the refolded protein preparation was adjusted to 3.0 using 5N HCL. The refolded protein preparation was initially concentrated to 1 gm/litre and further desalting was performed using exchange against 30 liter of 5 mM Glycine, pH 3.0 on a membrane having 5 kDa Molecular weight cut off (MWCO) to remove the refolding buffer components and denaturing agents.

### Example 4

### Effect of Redox Pair Concentration and Redox Pair Ratio on the Efficiency of Refolding of Human Proinsulin

The refolding percentage was calculated from the peak integration of the refolded Human proinsulin on HPLC with denatured, reduced preparation of proinsulin as a reference. The refolding buffer was consists of 100 mM Tris, 1 mM EDTA and varied concentration of refolding agents (Cysteine/Cystine). The protein concentration of Proinsulin was 0.3 mg/ml and the refolding reaction was carried out at 4° C. for 0.5 h. FIG. 4 shows the pH, shuffler concentrations used and the percentage efficiency of folding reaction. No significant difference in the refolding efficiencies was observed for all the different folding reactions carried out using varied concentration of cysteine/cystine. However, the maximum efficiency was seen for the reaction consisting of 3 mM Cysteine and 0.3 mM cystine at pH 9.5 (FIG. 4). The aliquots for the refolding reactions with varied concentrations of Cysteine and Cystine were collected after 30 minutes and the reaction was arrested with 0.1% Trifluoroacetic acid. 12 μg of protein containing aliquots were loaded on a 15% SDS gel under reducing and non reducing conditions. Silver staining of the gel was performed to analyze the refolding reaction. The SDS PAGE analysis shows a distinct mobility difference between the refolding aliquots loaded under reducing and non reducing condition. The Mobility difference is observed because of the compact nature of the molecule which is due to intact disulphide bonds under non-reducing conditions as compared to reducing conditions. The mobility difference observed is similar for all the aliquots loaded under reducing and non-reducing conditions (FIG. 5).

### Example 5

### Effect of Urea Concentration in the Refolding Buffer on the Efficiency of Refolding

Refolding reaction was carried out using varied concentration of urea (0.5 M-3 M) as a chaotropic agent in the refolding buffer. The refolding reaction was also carried out in the presence of two different concentrations of Cysteine/Cystine (5 mM Cystine/0.5 mM Cysteine and 0.3 mM Cystine/3 mM Cysteine) to evaluate the correlation of redox pair ratio and urea concentration on the refolding efficiency of protein. The aliquots of refolding preparation were taken for SDS PAGE analysis at interval of 30 min and the reaction was arrested with 0.1% Trifluro acetic acid. 12 μg of aliquots were loaded on a 15% SDS gel under reducing and non reducing conditions. The SDS PAGE analysis shows that as the urea concentration increases in the refolding system, it generates a constraint on the molecule which allows only partial disulphide bond formation, which can be confirmed from the presence of a heterogenic band. The SDS PAGE results show that as Urea concentration increases the heterogenicity becomes more prominent. The presence of heterogenicity (upper band observed in lane 12-15 in FIG. 6) is due to absence of disulphide bond which can be confirmed from the fact that a single band is generated for the aliquot taken from the refolding system consisting of 3 M urea, 0.3 mM Cysteine and 3 mM Cysteine and loaded under reducing conditions. The formation of more homogenous single band was observed for refolding system consisting up to 1.5 M urea (FIG. 6). Effect of urea concentration in the refolding buffer on the efficiency of refolding was also evaluated using RP-UFLC Analysis of aliquots of Human proinsulin preparation taken from refolding reaction carried out using refolding buffer consisting of 0.5 M-2.5M Urea. 10 μg of refolded proinsulin from every refolding buffer was injected on a C18 column and the reaction was monitored at 214 nm (FIG. 7). The refolding was found to be optimum at concentration of 1.5 M urea in the refolding buffer. A minute peak corresponding to trace amount of unfolded form of fusion protein linked Human proinsulin was observed (FIG. 7).

### Example 6

### Cloning and Expression of Recombinant Human Granulocyte Colony Stimulating Factor (rhG-CSF)

The cDNA of Human GCFS linked to a sequence coding for affinity tag (6× histidine) was cloned in pBR 322 derived expression vector and the vector was introduced into strain BL21 (DE3) of Escherichia coli. The fusion protein linked rhG-CSF sequence was expressed in the inclusion bodies of the E. coli using conventional methods. The cell pellet was collected by centrifugation at 4° C. and washed with buffer containing 20 mM Tris (pH 8.0), 0.5% Triton X-100 and 2M Urea and the inclusion bodies were collected by centrifugation. The pellets and supernatants obtained during Inclusion Bodies isolation process were analyzed using SDS PAGE.

### Example 7

### Pretreatment of Purified rhG-CSF

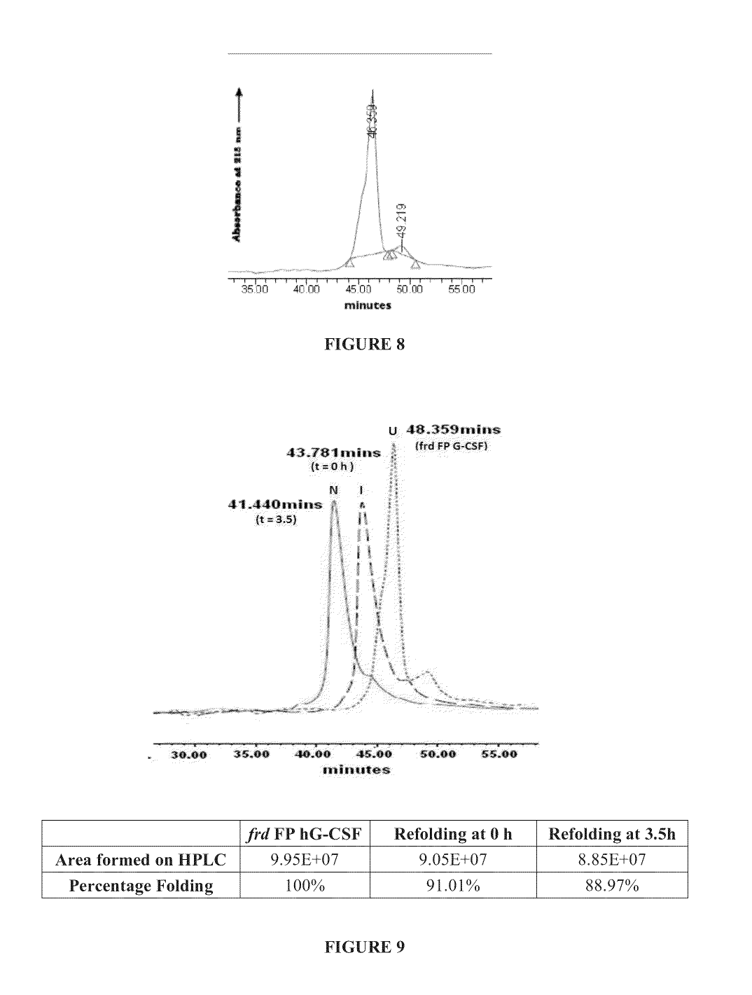

9 gm of isolated inclusion bodies were dissolved in 1 liter buffer containing 8.0 M Urea, 20 mM Tris (pH 8.5). The sample was then centrifuged at 8,000 rpm for 20 min at 4° C. The supernatant was collected and subjected to Immobilized Metal Ion Affinity Chromatography (IMAC) at room temperature. Fully reduced and denatured fusion protein preparation was prepared by adding the 3.5 gm of purified protein fraction of rhG-CSF from IMAC to a 1.0 liter of buffer containing 100 mM Tris-HCL (pH 8.5), 1 mM ethylene diamine tetraacetic acid (EDTA) and 100 mM Dithiothretol (DTT). The sample was incubated at 37° C. for three hours. 20 mM Glycine was added to the reduced and denatured protein preparation and the pH was adjusted to 3.0 using HCL. The low pH preparation was subjected to ultrafiltration using a membrane having a 5 kDa molecular weight cutoff (MWCO) against a 5 liter solution containing 8.0 M urea, 20 mM Glycine (pH 3.0) to remove the reducing agent. The low pH protein solution which was fully devoid of the reducing agent was analyzed using RP-HPLC (FIG. 8) to confirm the uniformity of the monomeric form of the protein. A sharp peak at 48.3 min was obsereved which corresponds to denature, reduced and exchanged Fusion protein linked to rhG-CSF.

### Example 8

### Folding of Fusion Protein Linked rhG-CSF

The low pH protein preparation (pH 3.0) which was treated to remove reducing agent is subjected to refolding in a 10 liter refolding buffer consisting of 100 mM Tris-HCL (pH 9.5), 1 mM EDTA, 3 mM of Cysteine, 0.3 mM of cystine and 1.5 M Urea. The refolding is performed at protein concentration of 0.3 mg/ml. The preparation is incubated at 4° C. for 0.5 h. The pH of the refolded protein preparation is adjusted to 8.5 using 5N NaOH. The desalting of the refolded protein preparation is performed using 55 liter volume (approximately 5-7× of buffer to be exchanged) of 10 mM Tris, pH 8.5 on a membrane having 5 kDa Molecular weight cut off (MWCO) to remove the refolding buffer components and denaturing agents.

### Example 9

### Reverse-Phase HPLC for Analyzing Refolding Reaction of Fusion Protein Linked rhG-CSF

Aliquots from the refolding reaction of recombinant Fusion Protein linked rhG-CSF (150 μg) were quenched by adding Trifluoroacetic acid to make pH 1.0 and injected onto a C4 (Symmetry 300TM C 4, Sum, 3.9×150 mm) reverse phase column. The solvents used for chromatography are Solvent A: 0.1%TFA; Solvent B: 0.1%TFA and 90% Acetonitrile. The column was initially equilibrated with 25% A at 1 ml/min. The separation is performed by a linear gradient from 25% B to 70% B within 50 minutes and from 70% B to 80% B over 5 min on WATERS 600E Multisolvent delivery system equipped with a photodiode array. Protein peaks are detected at 214 nm (FIG. 9). RP-HPLC analysis of refolding samples generated at different times for Fusion Protein linked rhG-CSF shows the formation of peak labeled ‘U’ corresponding to fully reduced and denatured Fusion Protein linked rhG-CSF dialyzed against 8 M urea, pH 3.0 and observed at 48.3 min. An intermediate peak (I) is observed at 43.7 min for the aliquot taken at 0 h. The completely folded native form (N) of the molecule is observed at 41.4 min for the aliquot taken at 3.5 h.

Presence of free Cysteine in G-CSF makes the refolding reaction complicated as the molecule has a tendency to adopt a pathway leading to aggregation. The area formed for fully reduced, denatured and exchanged fusion protein linked rhG-CSF is taken as 100% because the fully reduced and denatured fusion protein liked rhG-CSF (dialyzed against 8 M Urea, pH 3.0) does not have a tendency to get trapped in intermediate states and thus provides the complete area for the fusion protein. The area for the refolding reaction monitored at the 0 h and 3.5 h is found to be 9.05E+07 and 8.85E+07. The percentage efficiency for the refolding process was found to be 88.97%

What is claimed is:

1. A method for obtaining, a preparation of protein where substantial part of the protein is in a refolded form from a preparation comprising a mixture of protein in an aggregated, unfolded and misfolded form, the method comprising the sequence of steps of:
(i) adding a denaturing concentration of chaotropic agent to the first preparation comprising a mixture of protein in an aggregated, unfolded and misfolded form, to obtain a second preparation comprising the mixture of protein in a denatured form;
(ii) adding a reducing agent to the second preparation comprising the mixture of protein in a denatured form, incubating the said mixture for sufficient time at appropriate temperature to obtain a third preparation comprising the protein in a substantially monomeric form;
(iii) Lowering the pH of the said third preparation and removing the reducing agent in the presence of denaturing concentration of chaotropic agent while maintaining low pH to obtain a fourth preparation comprising the protein in a substantially monomeric form;
(iv) diluting the said fourth preparation with appropriate volume of refolding buffer of alkaline pH comprising a refolding agent and non denaturing concentration of chaotropic agent to obtain a fifth preparation where substantial part of the protein is refolded, and
(v) Separating the chaotropic agent and components of refolding buffer from said fifth preparation to obtain a desalted form of refolded protein preparation.

2. The method according to claim 1 wherein the reducing agent is selected from the group of Dithiothreitol (DTT), Cysteine, reduced glutathione, B-mercaptoethanol and other thiol group containing molecules.

3. The method according to claim 1 wherein the reducing agent is DTT.

4. The method according to claim 1 wherein the incubation time and incubation temperature of second preparation after addition of reducing agent is sufficient to form a third preparation comprising the protein in a substantially monomeric form.

5. The method according to claim 1 wherein the pH of the third preparation is lowered in the range of 1.5-3.5.

6. The method according to claim 1 wherein the removal of reducing agent from third preparation is carried out using dialysis, Ultra filtration, Size exclusion chromatography against low pH denaturing buffer devoid of reducing agent.

7. The method according to claim 1 wherein the refolding agent is selected from the group of cysteine/cystine, Reduced and oxidized glutathione, cysteamine/cystamine, DTT/GSSG and DTE/GSSG.

8. The method according to claim 1 wherein the pH of refolding buffer is in the range of 8.5-11.0.

9. The method according to claim 1 wherein the fourth preparation is diluted in refolding buffer to obtain concentration of 0.05 mg/ml-1 mg/ml.

10. The method according to claim 1 wherein the chaotropic agent is urea or Guanidine Hydrochloride.

11. The method according to claim 1 wherein the second preparation is optionally subjected to affinity chromatography.

12. The method according to claim 11 wherein the affinity chromatography is most preferably Immobilized Metal Ion Chromatography.

13. The method according to claim 1 wherein the denaturing concentration of chaotropic agent is in the range of 6M-10M for urea and 4-6M for Guanidine Hydrochloride.

14. The method according to claim 1 wherein the non-denaturing concentration of chaotropic agent is in the range of 0.1M-3 M for urea and 0.1-1.5M for Guanidine Hydrochloride.

15. The method according to claim 1 wherein the removal of chaotropic agent and components of the refolding buffer from the fifth preparation is carried out using dialysis, Ultra filtration, Size exclusion chromatography against a buffer of appropriate pH which is devoid of chaotropic agent and reducing agent.

16. The method according to claim 15 wherein the pH of the buffer is preferably basic if the fifth preparation consists of protein containing free SH groups.

17. The method according to claim 16 wherein the pH of the buffer is preferably acidic if the fifth preparation consists of protein devoid of free SH groups.

18. A method for obtaining, a preparation of protein which is devoid of any reducing agent and where substantial part of the protein is in an unfolded, monomeric form from a preparation comprising a mixture of protein in an aggregated, unfolded and misfolded form, the method comprising the sequence of steps of:
(i) adding a denaturing concentration of chaotropic agent to the first preparation comprising a mixture of protein in an aggregated, unfolded and misfolded form, to obtain a second preparation comprising the mixture of protein in a denatured form;
(ii) adding a reducing agent to the second preparation comprising the mixture of protein in a denatured form, incubating the said mixture for sufficient time at appropriate temperature to obtain a third preparation comprising the protein in a substantially monomeric form;
(iii) Lowering the pH of the said third preparation and removing the reducing agent in the presence of denaturing concentration of chaotropic agent while maintaining low pH to obtain a fourth preparation comprising the protein in a substantially monomeric form, wherein the protein solution obtained is substantially devoid of any reducing agent.

19. The method according to claim 18 wherein the second preparation is optionally subjected to affinity chromatography.

20. The method according to claim 19 wherein the affinity chromatography is most preferably Immobilized Metal Ion Chromatography.
